(19) United States
(12) Patent Application Publication
(10) Pub. No.: US 2023/0277804 A1
(43) Pub. Date: Sep. 7, 2023

(54) EMBOLIZATION MICROCATHETER FOR DELIVERY OF LARGE BEADS

(71) Applicant: ACCURATE MEDICAL THERAPEUTICS LTD., Rehovot (IL)

(72) Inventors: Tom DAGAN, Omer (IL); Yuval ZIPORY, Modiin (IL); Osnat HARBATER, Raanana (IL); Eran MILLER, Moshav Beit Elazari (IL)

(21) Appl. No.: 18/018,564

(22) PCT Filed: Jul. 27, 2021
(86) PCT No.: PCT/IL2021/050904

§ 371 Date: Jan. 29, 2023

(30) Foreign Application Priority Data
Jul. 30, 2020 (IL) 276416

(51) Int. Cl. A61M 25/00; A61M 25/01; A61B 90/00; A61B 17/12

(52) U.S. Cl. CPC A61M 25/0023; A61M 25/0108; A61M 25/007; A61B 90/39; A61B 17/12131; A61M 2025/0042; A61B 2090/3966

(57) ABSTRACT

An embolization microcatheter with a wall including a braid, a polymer formed around the braid and an inner liner coating an inner surface of the wall, a radiopaque marker located in proximity to the distal end opening; wherein the part of the microcatheter extending between the proximal end of the radiopaque marker and the distal end opening is devoid of the liner and wherein the wall has a thickness of about 120 microns or less.

### TECHNICAL FIELD

The present disclosure generally relates to the field of microcatheters for embolization, specifically to large diameter embolization catheters suitable for delivery through standard delivery catheters.

### BACKGROUND

Transarterial embolization therapy, tumor embolization, or transcatheter arterial embolization (TAE), involves administration of embolization material (which may include chemotherapeutics or/and radiotherapeutics) directly to a tumor (for example, liver tumors), via a microcatheter.

Embolization of tumors is typically performed utilizing microcatheters due to the requirement for selectively affecting the tumor while preventing, as much as possible, damage to healthy tissue. A major problem associated with embolization is “non-target embolization,” where the embolic material travels to blood vessels, other than those directly feeding the target tumor or tumor region, thus damaging healthy tissues, resulting in unpleasant and even hazardous outcomes. Possible scenarios include gastric ulcers caused by liver embolization, as well as cases where embolic material refluxes alongside the microcatheter reaching the wall of the stomach, possibly causing ischemia and ulceration. An additional phenomenon, which is abundant especially in advanced stage liver cancer, is non-target embolization through arterioportal shunt.

A microcatheter is usually passed via a larger-lumen catheter, which is placed within the proximal part of the vessel, such as the celiac or hepatic artery, and the microcatheter is then advanced therethrough towards the tumor until reaching a target location. In some scenarios, it is advantageous to use a diagnostic catheter as the delivery medium for the microcatheter. This procedure withholds the need of replacing one catheter with another, thus saving substantial time.

Another reason that microcatheters are routinely used in embolization procedures is the size of the feeding vessels, which carry blood directly to the organ or tumor. In order to reach as close as possible to the tumor, the embolization catheter is advanced into smaller and sometimes tortuous vessels. Accessibility to these vessels is difficult, if not precluded, with a larger and often stiffer catheter. Moreover, blood vessels in the body tend to go into spasm when manipulated, causing an ineffective embolic material delivery, so flexible micro-sized catheters are an absolute necessity.

A major drawback of trans-catheter embolization is that the embolization material, which is typically invisible, can be refluxed and reach non-target tissue and cause damage to them. In addition, reflux of embolization material may negatively affect the delivery of the embolization material to the target tissue, and thus impair treatment effectiveness and its clinical outcome.

Microcatheters with filter sections for delivery of embolization beads, while preventing backflow of the beads, have been disclosed by the inventors of the present applications. However, such catheters proved unsuitable for delivery of large embolization beads due to the beads clogging the microcatheter, while a simple enlarging of the outer diameter of the microcatheter makes them unsuitable for delivery through standard delivery catheters, such as 5.0 French delivery catheters.

### SUMMARY OF THE INVENTION

The present disclosure relates to embolization microcatheters which are suitable for delivery through 5.0 French delivery catheters, while also facilitating delivery of large embolization beads, i.e. beads with a particle size of 900 microns or more.

This is advantageously achieved by the unique structure of the wall of the embolization microcatheters.

The wall is made of polymeric material(s) formed around a braid which provide structural integrity to the microcatheter (especially along the filter section thereof) as well as trackability. The wall also includes an inner liner, coating the inner surface of the wall, and configured to reduce the friction between the wall and the beads flowing therethrough. Yet, the thickness of the wall is maintained below about 120 microns (at least along the portion thereof passing through the delivery microcatheter). The main obstacle to maintaining the thinness of the wall is the radiopaque marker located in proximity to the distal end opening of the microcatheter. It was surprisingly found by the inventors of the hereindisclosed microcatheters that the desired wall thinness may be accomplished and maintained, by having the inner liner extending only up until the radiopaque marker so as to avoid bulking, and that absence of a liner along the part of the microcatheter extending from the radiopaque marker to the distal end opening, has minimal to no influence on the delivery of the beads through the distal end opening.

The hereindisclosed embolization microcatheters may further include a filter section with a plurality of openings configured for outflow of fluids while preventing outflow of the beads, thus ensuring concentrated delivery of the embolization beads with minimal backflow.

Advantageously, the size, shape and distribution of the openings enables smooth delivery of the beads, despite their large size. This is accomplished by including two or more filter segments, each filter segment comprising a plurality of side openings having the proximal most of the circumferential sections distributed circumferentially around the microcatheter, wherein the proximal most of the circumferential sections includes fewer side openings than sections distal thereto. As a result, the outflow of fluid is relatively low at the proximal end of the filter, thus reducing the chance of clogging, and increases toward the distal end opening at which a low outflow rate is desired in order to prevent backflow. In addition, the shape and size of the openings change between the proximal end and the distal end of the filter in that the distal most part of the filter includes small, preferably square-shaped openings configured to prevent the beads to get stuck and/or sucked in as the amount of suspension fluid is reduced.

Some aspects of the present disclosure provide an embolization microcatheter comprising: an elongated tubular member forming a lumen, the elongated tubular member terminating with a distal end opening, wherein a wall of the elongated tubular member comprises a braid, a polymer formed around the braid and an inner liner coating an inner surface of the wall; wherein the wall of the part of the elongated tubular member extending between a proximal radiopaque marker and the distal end opening has a thickness of less than or equal to 130 microns; and a distal radiopaque marker located in proximity to the distal end opening; wherein the part of elongated tubular member extending between the proximal end of the radiopaque marker and the distal end opening is devoid of the liner.

According to some embodiments, the outer diameter of the elongated tubular member is less than or equal to 1.5 mm and an inner diameter of the elongated tubular member is 700 microns or more.

According to some embodiments, the thickness of the distal end of the wall is less than or equal to 120 microns.

According to some embodiments, the embolization microcatheter includes a filter formed in the wall of the elongated tubular member, the filter comprising two or more filter segments, each filter segment comprising a plurality of side openings penetrating the wall, the plurality of side openings distributed circumferentially around the elongated tubular member.

According to some embodiments, the proximal most of the filter segments comprises fewer side openings than rings distal thereto.

According to some embodiments, the distal most of the filter segment is positioned 2-4 mm proximally to the distal end opening.

According to some embodiments, the side openings of the distal most filter segment are essentially square shaped. According to some embodiments, the essentially square shaped side openings have a dimension of 50×50 microns. According to some embodiments, the essentially square shaped side openings have a cross-sectional dimension of 80×50 microns.

According to some embodiments, the side openings of the remainder of the at least two filter segments are in a form of axial slits.

According to some embodiments, the side openings of the remainder of the at least two filter segments have a width of 20 microns and wherein the length of the side openings of the remainder of the at least two filter segments varies between the side openings.

According to some embodiments, the side openings of the remainder of the at least two filter segments have a length of at least 200 microns.

According to some embodiments, at least some of the side openings of the remainder of the at least two filter segments have dimension of 2700×20 microns.

According to some embodiments, at least some of the side openings of the remainder of the at least two filter segments have a dimension of 1350×20 microns. According to some embodiments, at least some of the side openings of the remainder of the at least two filter segments have a dimension of 300×20 microns.

According to some embodiments, the braid is made of tungsten.

According to some embodiments, the inner liner comprises Polytetrafluoroethylene (PTFE).

According to some embodiments, the radiopaque marker comprises a metal marker band. According to some embodiments, the radiopaque marker is located 0.5-2 mm proximally to the distal end opening.

According to some embodiments, the embolization microcatheter is suitable for delivery of embolization beads having a particle size of about 900 microns.

According to some embodiments, the embolization microcatheter has a length of at least 1 m.

According to some embodiments, there is provided a method for delivering embolization beads, the method comprising: delivering the hereindisclosed embolization microcatheter to a target location via a delivery catheter; and injecting beads through the microcatheter.

According to some embodiments, the beads have a particle size of at least 500 microns. According to some embodiments, the beads have a particle size of at least 900 microns.

According to some embodiments, the delivery catheter is a 5.0 Fr delivery catheter.

Certain embodiments of the present disclosure may include some, all, or none of the above characteristics. One or more technical advantages may be readily apparent to those skilled in the art from the figures, descriptions and claims included herein. Moreover, while specific characteristics have been enumerated above, various embodiments may include all, some or none of the enumerated characteristics.

In addition to the exemplary aspects and embodiments described above, further aspects and embodiments will be further expanded upon in the figures and the following detailed descriptions.

### BRIEF DESCRIPTION OF THE FIGURES

The features, nature and advantages of the present disclosure will become more apparent from the detailed description set forth below when taken in conjunction with the drawings in which like reference characters identify correspondingly throughout. Identical structures elements or parts that appear in more than one figure are generally labeled with the same number in all the figures in which they appear. Alternatively, elements or parts that appear in more than one figure may be labeled with different numbers in the different figures in which they appear. The dimensions of the components and features in the figures were chosen for convenience and clarity of presentation and are not necessarily shown to scale. The figures are listed below.

FIG. 1A schematically illustrates a microcatheter comprising an outer layer including a plurality of sections, the plurality of sections made of different polymeric materials, according to some embodiments;

FIG. 1B schematically illustrates a perspective, cutaway view of the distal end of the microcatheter of FIG. 1A illustrating the outer layer, the strike layer, the inner layer, the braided skeleton located between the inner layer and the outer layer.

FIG. 2A schematically illustrates a 3.0 Fr embolization microcatheter with a fluid barrier forming section, according to some embodiments.

FIG. 2B schematically illustrates a magnified and partially exposed view of the distal end of the microcatheter of FIG. 2A, according to some embodiments.

FIG. 2C schematically illustrates a magnified and partially exposed view of the distal tip of the microcatheter of FIG. 2A, according to some embodiments.

FIG. 2D schematically illustrates a slit formed by selective cutting through the wall of a fluid barrier forming section, such as the fluid barrier forming section of the embolization microcatheter of FIG. 2A, according to some embodiments.

FIG. 3 schematically illustrates an optional slit pattern of the microcatheter of FIG. 2A.

FIG. 4 schematically illustrates another optional slit pattern of the microcatheter of FIG. 2A.

### DETAILED DESCRIPTION OF THE INVENTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. However, it will also be apparent to one skilled in the art that these concepts may be practiced without specific details being presented herein. In some instances, well-known features may be omitted or simplified in order to avoid obscuring the disclosure.

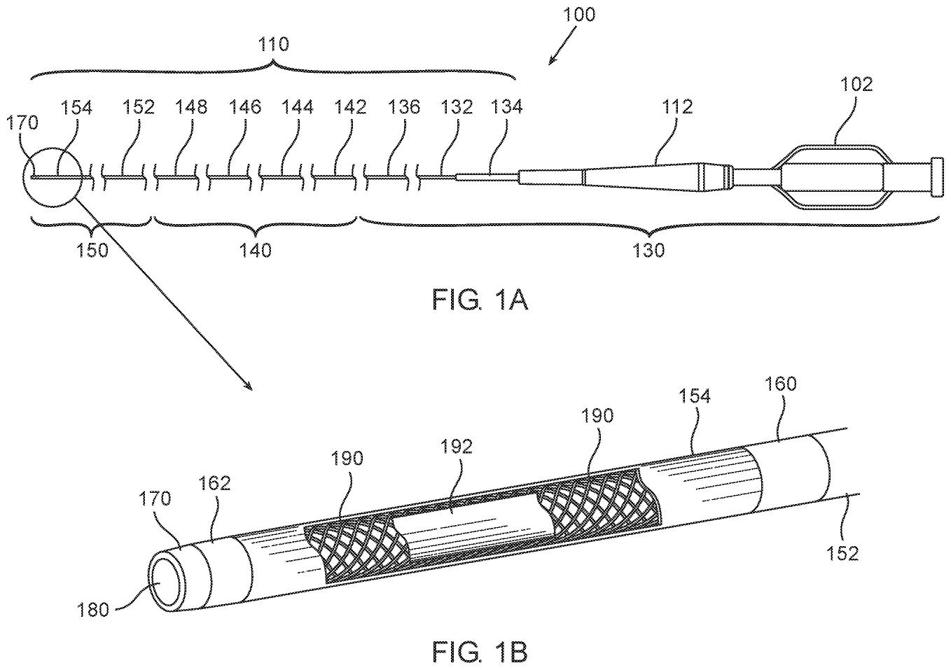

Reference is now made to FIG. 1A, which schematically illustrates an embolization microcatheter 100, and to FIG. 1B which illustrates magnified/exposed views of a distal part of FIG. 1A.

As used herein, the terms “embolization”, “transcatheter embolization”, “transcatheter arterial embolization” and “TAE” may be used interchangeably and refer to the passage and lodging of an embolus within the bloodstream for therapeutic purposes, for example, as a hemostatic treatment of bleeding or as a treatment for some types of cancer by deliberately blocking blood vessels to starve the tumor cells.

Embolization microcatheter 100 is a 3.0 Fr microcatheter having an elongated tubular member 110 with an outer diameter of about 1.5 mm, or an outer diameter in the range of 1.0-1.5 mm, or 1.2-1.6 mm and an inner diameter in the range of 700-850 microns.

The proximal end 130 of microcatheter 100 includes a hub 102 which is molded on or otherwise attached to elongated tubular member 110 of microcatheter 100.

Hub 102 is configured to allow access to the lumen of elongated tubular member 110 for a variety of functions, such as the injection of fluids or drugs, or the introduction of guidewires. Hub 102 includes a strain relief 112, preferably mechanically coupled to hub 102. Strain relief 112 may be made of a polymeric material and may, as illustrated, be tapered at its distal end. Strain relief 112 can be configured to provide structural support to elongated tubular member 110, to prevent it from kinking.

According to some embodiments, the wall of elongated tubular member 110 may include a plurality of sections, each section characterized by the polymers utilized. According to some embodiments, a plurality of sections may include 3, 4, 5, 6, 7, 8, 9, 20 or more sections. Each possibility is a separate embodiment.

According to some embodiments, the different polymeric layers may contribute to different characteristics of the layer/section and thus of elongated tubular member 110. For example, the different polymeric layers may contribute to the elasticity, flexibility, stretch-ability, strength, hardness, rigidity, ultimate tensile strength, elongation or any other characteristic of the layer and thus the microcatheter. Each possibility is a separate embodiment.

The proximal end 130 of elongated tubular member 110, attached to strain relief 112, includes an outer layer 132 made of a polyether block amide having a hardness of about 70D shore and/or a flexural modulus of about 74,000 psi. According to some embodiments, proximal end 132 may have a length of 600-1200 mm (e.g. about 1000 mm).

Optionally, part of outer layer 132 may include a heat shrink material 134 covering the joint between strain relief 112 and elongated tubular member 110.

Adjacent section 132, is a second section 136 of elongated tubular member 110, made of a polyether block amide having a hardness of about 60D-65D shore and/or a flexural modulus of about 41,000 psi. Section 136 may have a length of 20-60 mm, e.g. 40 mm.

Intermediate part 140 of elongated tubular member 110 includes section 142 having an outer layer which may be made of a polyether block amide or other suitable polymer having a hardness of about 55D shore and/or a flexural modulus of about 25,000 psi, section 144 having an outer layer made of a polymeric material having a hardness of about 40D shore such as a polycarbonate-based thermoplastic urethane having a hardness of about 40D shore, and/or a flexural modulus of about 10,000-12,000 psi; section 146 having an outer layer made of a one or more polycarbonate-based thermoplastic urethanes having a hardness of about 95A shore, and section 148 having an outer layer made of a one or more polycarbonate-based thermoplastic urethanes having a hardness of about 85A shore.

Section 142 may have a length of about 30 to about 80 mm, e.g. 40 mm. Section 144, may have a length of about 30 mm to about 70 mm (e.g. about 50 mm). Section 146 may have a length of about 80 mm to about 150 mm (e.g. about 130 mm). Section 148 may have a length of about 20 mm to about 60 mm (e.g. about 45 mm).

According to some embodiments, the distal end may refer to the distal 100 mm, 50 mm, 30 mm, 20 mm, 15 mm, 10 mm, 5 mm or 2 mm of microcatheter 100. Each possibility is a separate embodiment. According to some embodiments, the distal end may refer to the part of the microcatheter extending between the proximal marker and distal end opening 180.

According to some embodiments, the polymeric material of all sections in the intermediate part 140 is softer than that of the polymeric material of the wall section in proximal end 130 of tubular member 110.

According to some embodiments, the polymeric material of section 148 is softer than that of section 146. According to some embodiments, the polymeric material of section 146 is softer than that of section 144. According to some embodiments, the polymeric material of section 144 is softer than that of section 142.

Distal end 150 of elongated tubular member 110 includes sections 152 having an outer layer made of a polymeric material having a hardness of about 85A shore such as a polycarbonate-based thermoplastic urethane having a hardness of about 85A shore and section. The polymer of section 152 may further include a polymeric radiopaque marker, such as but not limited to tantalum powder; section 154 having an outer layer made of a polymeric material having a hardness of about 95A shore such as a polycarbonate-based thermoplastic urethane having a hardness of about 95A shore. According to some embodiments, the distal tip 170 of elongated tubular member 110 terminates with a distal end opening. According to some embodiments, the distal tip refers to the distal marker 162 and distal end opening 180.

As used herein, the term “distal end opening” refers to the end opening of the microcatheter leading into the lumen thereof. According to some embodiments, distal end opening 180 defines the termination of the microcatheter at the distal end thereof. According to some embodiments, distal end opening 180 may have an inner diameter essentially equal to the inner diameter of the microcatheter lumen. According to some embodiments, the distal end opening 180 may have an inner diameter which is smaller than the inner diameter of the microcatheter lumen leading to a narrowing of the lumen toward the end thereof.

Section 152 includes a proximal marker 160 (seen in FIG. 1B), section 154 terminates with a distal marker 162 (also seen in FIG. 1B). According to some embodiments, distal end 150 may have a length of 10-20 mm. According to some embodiments, section 152 may have a length of 2-10 mm (e.g. about 6 mm). According to some embodiments, section 154 may have a length of 5-20 mm (e.g. about 10 mm). According to some embodiments, proximal marker 160 may be a radiopaque powder embedded in part of outer layer of section 152, as essentially described herein. According to some embodiments, proximal marker 160 may be positioned approximately 5-20 mm, or 10-15 mm from the distal end opening 180. According to some embodiments, distal marker 162 may be a radiopaque alloy submerged in outer layer 154. According to some embodiments, distal marker 162 may be positioned approximately 1 mm proximally from distal end opening 180.

Reference is now made to FIG. 1B which schematically illustrates a perspective, cutaway view of the distal part of distal end 150 of microcatheter 100 shown in FIG. 1A extending from proximal marker 160 to a tip 170 and encompassing section 154 of elongated tubular member 110. As stated above, the wall of section 154. As seen from the exploded view, underneath the outer layers is a braid 190.

According to some embodiments, braid 190 extends along the entire length of tubular member 110. Alternatively, braid 190 extends along only a portion of elongated tubular member 110, such as only along section 154, along sections 152 and 154, along sections 152, 154 and 148, along sections 152, 154, 148 and 146, along sections 152, 154, 148, 146 and 144, or along sections 152, 154, 148, 146, 144 and 142. Each possibility is a separate embodiment.

According to some embodiments, braid 190 has a picks-per-inch (PPI) value ensuring that, in combination with a low durometer polymer, a flexible distal end is obtained, and in combination with a polymer having a higher durometer a relatively stiff proximal end is provided. According to some embodiments, the braided skeleton may have a wire arrangement of 75-150 Picks Per Inch (PPI), 100-150 PPI or 100-150 PPI. Each possibility is a separate embodiment. As a non-limiting example, the braided skeleton may have a wire arrangement of about 140 PPI or about 145 PPI. Those skilled in the art will appreciate the term picks-per-inch (PPI) is a measurement of braid wire density and represents the number of picks (e.g. weft wires) per inch of braid.

As used herein the terms “braid” and “braided skeleton” may refer to a structural element, such as a tubal element formed of a plurality of interlaced wires. According to some embodiments, the braid may be formed of at least three interlaced wires forming a tube. According to some embodiments, the braid may include 8-48 wires or 12-32 wires. As a non-limiting example, the braid may include 16 wires. Each possibility is a separate element. According to some embodiments, the wires forming the braid may have a diameter in the range of 10-60 microns such as 15-40 microns or 20-30 microns or any other suitable diameter within the range of 10-60 microns. Each possibility is a separate embodiment. As a non-limiting example, the wires forming the braid may have a diameter of 25 microns. According to some embodiments, the skeleton may extend along essentially the entire length of the catheter. According to some embodiments, the braid may be made from tungsten, stainless steel, Nickel titanium (also referred to as Nitinol), nitinol, cobalt chrome, platinum iridium, nylon or any combination thereof. Each possibility is a separate embodiment.

According to some embodiments, at least some of the wires forming the braided skeleton may be braided in a same or opposite direction, i.e. left/right handed. Advantageously, the braiding structure allows good torque-ability (better than a coiled skeleton), low flexural rigidity (i.e. good flexibility), good push-ability (better than a coiled skeleton), and superior kink-resistance.

According to some embodiments, at least some of the wires forming the braided skeleton may be non-circular/round.

Underneath braid 190 is an inner liner 192, which may be made of Polytetrafluoroethylene (PTFE). According to some embodiments, inner liner 192 may have a thickness of 10-30 micron or 10-25 micron. Each possibility is a separate embodiment. According to some embodiments, the liner is a film cast liner.

Advantageously, in order to compensate for the bulkiness distal marker 162, the part of elongated tubular member 110 extending from the distal end of the distal marker 162, to distal end opening 180 is devoid of inner liner 192, thereby essentially maintaining the inner and outer diameters of elongated tubular member 110, despite the bulkiness of distal marker 162, which enables smooth delivery through a 5.0 Fr delivery catheter (not shown).

According to some embodiments, the total thickness of the wall of the distal end of the elongated tubular member does not exceed about 130 microns, at least along the part of the elongated tubular member to be inserted through the 5.0 Fr delivery catheter. According to some embodiments, the total thickness of the wall of the distal end of the elongated tubular member does not exceed about 120 microns, at least along the part of the elongated tubular member to be inserted through the 5.0 Fr delivery catheter.

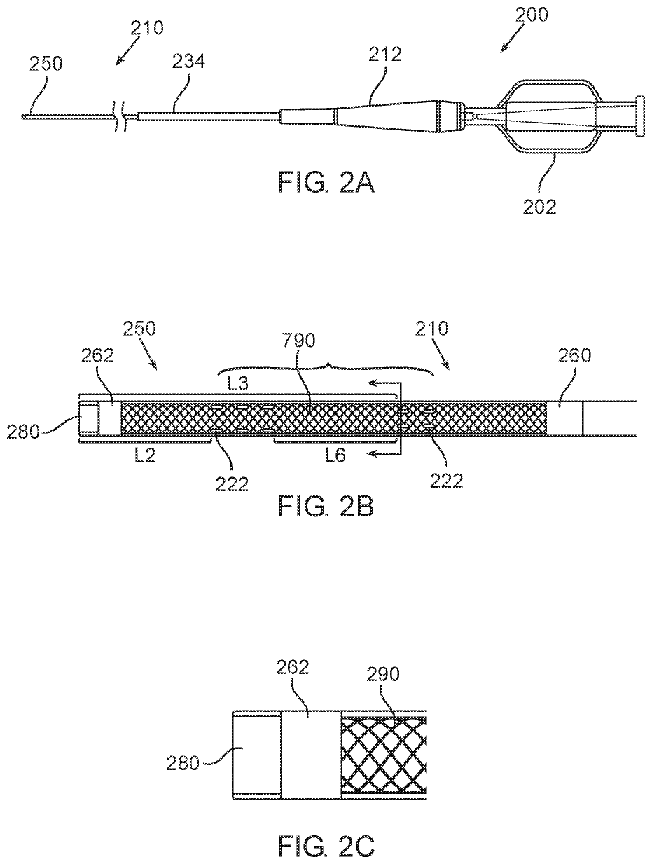

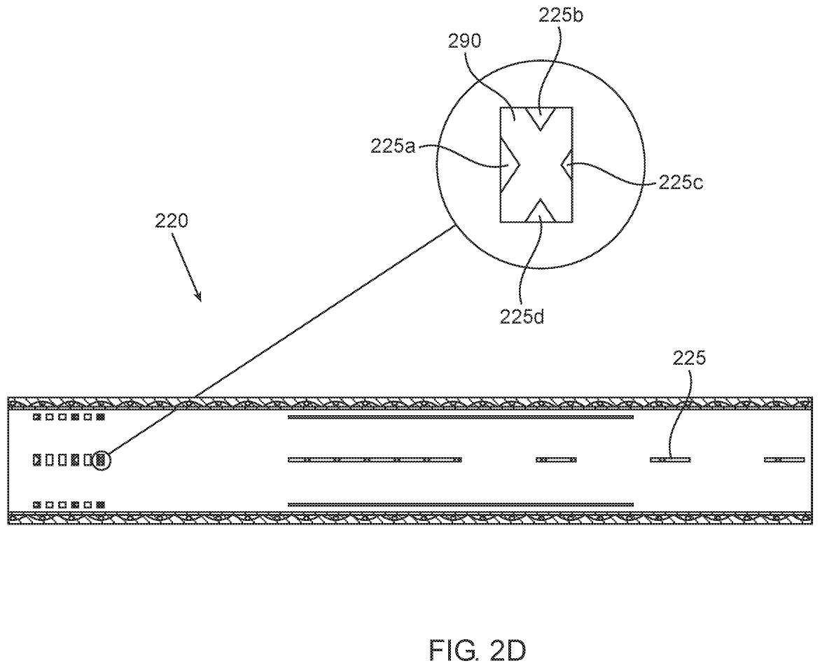

Reference is now made to FIG. 2A-FIG. 2C, which schematically illustrate an embolization microcatheter 200 and magnified/exposed views of parts thereof. Embolization microcatheter 200 may be similar to embolization microcatheter 100 apart from embolization microcatheter 200 also including a filter 220. Reference is also made to FIG. 2D, which schematically illustrates the structure of filter 220. Embolization microcatheter 200 is a 3.0 Fr microcatheter having an elongated tubular member 210 with an outer diameter in the range of about 1.0-1.5 mm, or in the range of about 1.2 mm-1.6 mm and an inner diameter in the range of 700-850 microns. Embolization microcatheter 200 is particularly suitable for delivery of large embolization beads, such as but not limited to embolization beads having an average particle size of 500 microns or more, 600 microns or more, 700 microns or more, 800 microns or more, 900 microns or more and 1000 microns or more. Each possibility is a separate embodiment.

Filter 220 including a plurality of penetrating side openings formed in the wall of elongated tubular member 210, is schematically illustrated in FIG. 2B.

As used herein, the term “plurality” with reference to the side openings refers to 2 or more, 3 or more, 5 or more, 10 or more, 15 or more, 20 or more or 25 or more axial slits. Each possibility is a separate embodiment.

According to some embodiments, the filter 220 may be an integral part of elongated tubular member 110 and may extend along a length of 0.3 mm-20 mm, such as 1 mm-10 mm, 1 mm-5 mm, 1.5 mm-5 mm, 2 mm-5 mm or any other in-between suitable length. Each possibility is a separate embodiment.

According to some embodiments, filter 220 may have a total open area, formed by the side openings, in the range of 0.2-1 mm², 0.2-0.6 mm², 0.3-1 mm², 0.3-0.5 mm², 0.4-0.6 mm², 0.5-1.5 mm², 1.0-3.5 mm², 1.5-4 mm², 2.0-3.5 mm² or any other suitable area within the range of 0.1-4 mm². Each possibility is a separate embodiment. According to some embodiments, at least 5%, at least 10%, or at least 15% of filter 220 is open area formed by the side openings. According to some embodiments, 5%-30%, at least 7%-25%, 7%-20%, 5%-15% of filter 220 is open area formed by the side openings. Each possibility is a separate embodiment.

According to some embodiments, the side openings may be formed by selective cutting (e.g. selective laser cutting), that is, without cutting the wires forming braid 290 as illustrated in FIG. 2D. According to some embodiments, the part of the liner positioned below the wires remains intact. According to some embodiments, both the polymeric layer and the inner liner positioned between the wires of braid 290 are penetrated when forming the slits. Advantageously, the selective cutting of the polymeric layer (leaving braid 290 essentially intact may provide subdivision of at least some of the side-openings into two or more sub-side-openings (here sub-side-opening 225a-225d) separated by the braid but not by the polymeric outer layer.

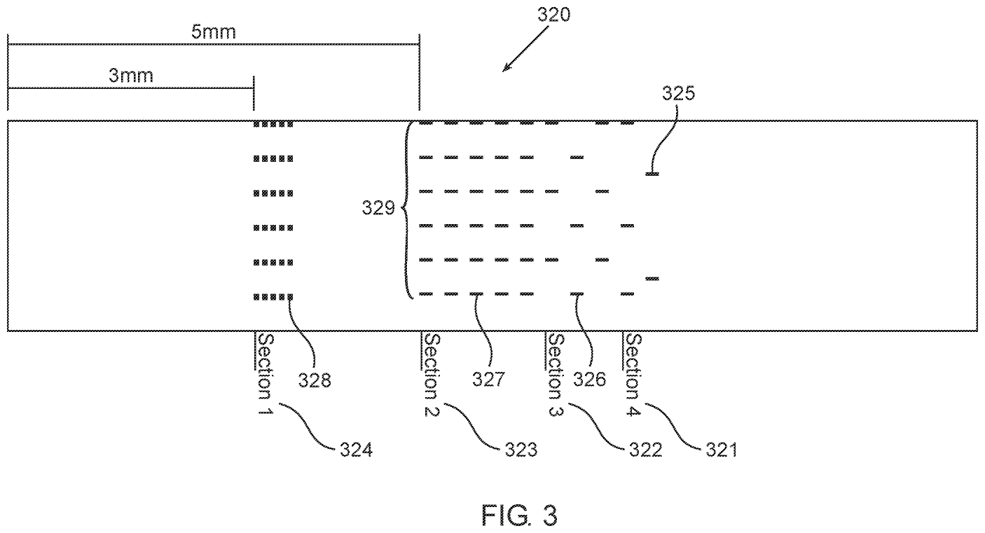

One optional structure of filter 220 is provided in FIG. 3. As seen in FIG. 3, filter 320 may include four filter sections 321, 322, 323 and 324 each filter section comprising a plurality of side openings 325, 326, 327 and 328, distributed in circumferential rings around filter member 320.

According to some embodiments, the proximal most filter section, filter section 321 has fewer side openings than filter sections distal thereto. According to some embodiments, filter section 321 may include 1-4 or 1-3 rings of side openings, such as, but not limited to 2 rings of side opening. According to some embodiments, each of the rings may include 1-5 side openings or 1-3 side openings, such as, but not limited to 2 side openings per ring. According to some embodiments, filter section 321 may include a total of 1-5, 2-4 side openings, such as but not limited to 2 side openings.

According to some embodiments, side openings of the proximal most circumferential sections, are circumferentially shifted relative to side openings in its neighboring circumferential section.

According to some embodiments, side openings 325 in a first ring of filter section 321 may be circumferentially shifted relative to side openings in its neighboring ring and/or circumferentially shifted relative to side openings 326 of filter section 322.

According to some embodiments, filter section 322 may include 1-5 or 2-4 rings of side openings, such as, but not limited to 3 rings of side opening. According to some embodiments, each of the rings may include 1-5 side openings or 2-4 side openings, such as, but not limited to 3 side openings per ring. According to some embodiments, filter section 322 may include a total of 5-15, 6-10 side openings, such as but not limited to 9 side openings.

According to some embodiments, side openings 326 in a first ring of filter section 322 may be circumferentially shifted relative to side openings in its neighboring ring.

According to some embodiments, filter section 323 may include 2-10 or 3-7 rings of side openings, such as, but not limited to 5 rings of side opening. According to some embodiments, each of the rings may include 2-15 side openings or 4-8 side openings, such as, but not limited to 6 side openings per ring. According to some embodiments, filter section 323 may include a total of 10-50, 20-40 side openings, such as but not limited to 30 side openings.

According to some embodiments, side openings 325, 326 and 327 of filter sections 321, 322 and 323 respectively may be essentially rectangularly shaped axial slits. Due to the shape of the filter 220, side openings 325, 326 and 327 may be conical, i.e. have a larger cross-section at the outer surface than at the inner surface of filter 220.

The sizes provided below relate to measures made at the inner surface.

According to some embodiments, side openings 325, 326 and 327 may have a dimension of about 150×20 microns.

According to some embodiments, each ring of side openings 325, 326 and 327 of filter sections 321, 322 and 323 may be spaced apart from its neighboring ring by 100-200 microns or by 120-180 microns, such as but not limited to 150 microns.

According to some embodiments, the distal most of filter section, filter section 324 is positioned about 2-5 mm proximally to the distal end opening. According to some embodiments, the distal most of filter section, filter section 324 is positioned about 3 mm proximally to the distal end opening.

According to some embodiments, the side openings 328 of filter section 324 are essentially square shaped. Advantageously, the square-shaped size of the side openings 328 enables outflow of fluid, while ensuring minimal interference to the flow of beads towards distal end opening, despite the beads already being concentrated due to the outflow of fluid through side openings 325, 326 and 327. According to some embodiments, filter section 324 may include 3-10 or 4-6 rings of side openings, such as, but not limited to 5 rings of side opening. According to some embodiments, each of the rings may include 3-10 side openings or 4-8 side openings, such as, but not limited to 6 side openings per ring.

According to some embodiments, side openings 328 of filter section 324 may have a dimension of about 50×50 microns.

According to some embodiments, the distal most ring of side openings 328 may be spaced apart by 1-10 mm or 2-7 mm, such as but not limited to 2 mm from a distal most of the rings of side openings 327 in filter section 323.

According to some embodiments, the distal most ring of side openings 328 may be spaced apart by 1-10 mm or 2-5 mm, such as but not limited to 3 mm from the distal end opening.

According to some embodiments, each ring of side openings 328 of filter section 324 may be spaced apart from its neighboring ring by 20-100 microns or by 30-60 microns, such as but not limited to 50 microns.

According to some embodiments, the slits may be positioned at a same or a different longitudinal position. Each possibility is a separate embodiment. According to some embodiments the distribution of the slits may be staggered, zig-zagged or any other suitable even or uneven distribution.

Advantageously, the filter 320 may be configured for kink-free bending despite the plurality of slits formed in the wall thereof. According to some embodiments, the flexibility of the filter 220 is determined by the number of side openings, their minimal cross-sectional dimension, their width, length spacing, geometry, distance from distal outlet etc., as essentially described herein, may enable kink-free bending thereof.

As used herein the term “kink-free bending” may refer to a bending of filter 320, which does impede flow therethrough. According to some embodiments, filter 320 may be configured for kink-free bending at an angle of about 180 degrees. According to some embodiments, filter 320 may be configured for kink-free bending at a minimum bending radius in the range of about 0.5 to 1.5 mm, for example 0.5 to 1.2, 0.5 to 1 mm, or any radius in-between.

Advantageously, microcatheter 300 including filter 320 provides effective reflux prevention, which requires a relatively high density of side openings, while a small kink-free radius (e.g. in the range of 0.5 to 1.5 mm) and tensile strength of at least 5N is still ensured.

According to some embodiments, the microcatheter 300 may have a length of at least 50 cm, at least 60 cm, at least 75 cm, or at least 1 m. Each possibility is a separate embodiment.

According to some embodiments, the outer wall of the microcatheter 300 may be non-tapered essentially along its/their entire length.

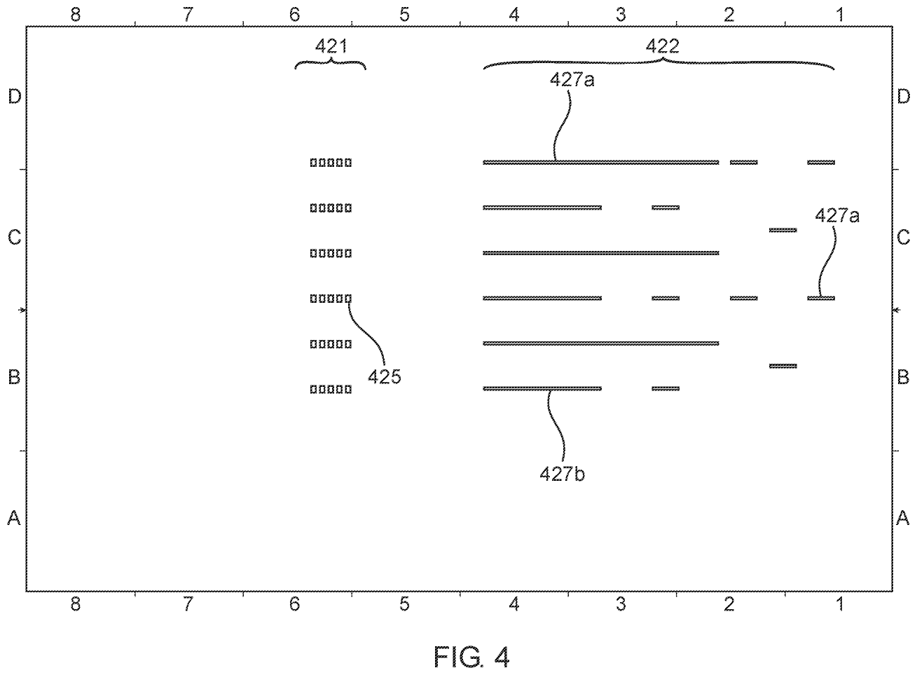

One optional structure of filter 220 is provided in FIG. 4. As seen in FIG. 4, filter 420 may include two filter sections 421 and 422 each filter section comprising a plurality of side openings 425 and 427, distributed in circumferential rings around elongated tubular filter 420.

Due to the shape of the filter 220, side openings side openings 425 and 427 may be conical, i.e. have a larger cross-section at the outer surface than at the inner surface of filter 220.

The sizes provided below relate to measures made at the inner surface.

According to some embodiments, filter section 421 may include 2-10 or 2-6 annular rings of side openings, such as, but not limited to 5 rings of side opening. According to some embodiments, each of the rings may include 1-10 side openings or 2-8 side openings, such as, but not limited to 6 side openings per ring. According to some embodiments, filter section 421 may include a total of 10-60 side openings or 20-40 side openings, such as but not limited to 30 side openings. According to some embodiments, side openings 425 may be essentially square or rectangularly shaped. According to some embodiments, side openings 425 may have a width of 50-150 microns or 50-100 microns such as e.g. about 80 microns. According to some embodiments, side openings 425 may have a length of 20-100 microns or such as e.g. about 50 microns. According to some embodiments, each of the rings of side opening may be spaced apart longitudinal from its neighboring ring by 20-100 microns, e.g. by approximately 50 microns. According to some embodiments, the distal most of side openings 425 may be positioned about 2-5 mm e.g. about 3 mm, from the distal end opening of the microcatheter.

Advantageously, the square-shaped size of the side openings 425 enables outflow of fluid while ensuring minimal interference to the flow of beads towards distal end opening, despite the beads already being concentrated due to the outflow of fluid through side openings 325, 326 and 327.

According to some embodiments, filter section 422 may include 5-50 or 10-30 or 10-20 side opening 427. According to some embodiments, the width of side openings 427 may by in the range of 10-50 or 10-30 microns, e.g. about 20 microns. According to some embodiments, at least some of side opening 427 may have a different length. According to some embodiments, at least some of side opening 427, such as side opening 427a may have a length in a range of about 1 mm-4 mm or about 2 mm-3.5 mm, e.g. about 2.7 mm. According to some embodiments, at least some of side opening 427, such as side opening 427b may have a length in a range of about 0.8 mm-2 mm or about 1 mm-1.5 mm, e.g. about 1.35 mm. According to some embodiments, at least some of side opening 427, such as side opening 427c may have a length in a range of about 0.2 mm-1 mm or about 0.2 mm-0.5 mm, e.g. about 0.3 mm. Advantageously, filter 220 may be formed by selective cutting of the polymeric layer (leaving braid 290 essentially intact). According to some embodiments, at least some of side openings 427 may include sub-side-openings (illustrated as with numbers 225a-225d in FIG. 2D) separated by braid 290, but not by the polymeric outer layer.

According to some embodiments, each of side openings 427 is spaced apart longitudinally from a neighboring side opening of side opening may be spaced apart from its neighboring side opening by 20-200 microns. According to some embodiments, the longitudinal spacing between side openings 427 may vary. According to some embodiments, at least some of side openings 427 are axially shifted vis-à-vis a neighboring side opening. According to some embodiments, the distal most of side openings 427 may be positioned about 3-10 mm e.g. about 5 mm, from the distal end opening of the microcatheter.

According to some embodiments, filter 420 may have a length of 5-15 mm or about 5-10 mm.

According to some embodiments, the slits may be positioned at a same or at a different longitudinal position. Each possibility is a separate embodiment. According to some embodiments the distribution of the slits may be staggered, zig-zagged or any other suitable even or uneven distribution.

Advantageously, the filter 420 may be configured for kink-free bending despite the plurality of slits formed in the wall thereof. According to some embodiments, the flexibility of the filter 220 is determined by the number of side openings, their minimal cross-sectional dimension, their width, length spacing, geometry, distance from distal outlet etc., as essentially described herein, may enable kink-free bending thereof.

As used herein the term “kink-free bending” may refer to a bending of filter 420, which does impede flow therethrough. According to some embodiments, filter 420 may be configured for kink-free bending at an angle of about 180 degrees. According to some embodiments, filter 420 may be configured for kink-free bending at a minimum bending radius in the range of about 0.5 to 1.5 mm, for example 0.5 to 1.2, 0.5 to 1 mm, or any radius in-between.

Advantageously, microcatheter 400 including filter 420 provides effective reflux prevention, which requires a relatively high density of side openings, while a small kink-free radius (e.g. in the range of 0.5 to 1.5 mm) and tensile strength of at least 5N is still ensured.

According to some embodiments, the microcatheter 400 may have a length of at least 50 cm, at least 60 cm, at least 75 cm, or at least 1 m. Each possibility is a separate embodiment. Each possibility is a separate embodiment.

According to some embodiments, the outer wall of the microcatheter 400 may be non-tapered essentially along its/their entire length.

As used herein, the terms “approximately” and “about” refer to +/−10%, or +/−5%, or +−2% vis-à-vis the range to which it refers. Each possibility is a separate embodiment.

While a number of exemplifying aspects and embodiments have been discussed above, those of skill in the art will envisage certain modifications, additions and sub-combinations thereof. It is therefore intended that the following appended claims and claims hereafter introduced be interpreted to include all such modifications, additions and sub-combinations as are within their true spirit and scope.

1. An embolization microcatheter comprising:
an elongated tubular member forming a lumen, the elongated tubular member terminating with a distal end opening, wherein a wall of the elongated tubular member comprises a braid, a polymer formed around the braid and an inner liner coating an inner surface of the wall; wherein the wall of the part of the elongated tubular member extending between a proximal radiopaque marker and the distal end opening has a thickness of less than or equal to 130 microns; and
a distal radiopaque marker located in proximity to the distal end opening;
wherein the part of elongated tubular member extending between the proximal end of the distal radiopaque marker and the distal end opening is devoid of the liner

2. The embolization microcatheter according to claim 1, wherein an outer diameter of the elongated tubular member is less than or equal to 1.5 mm and an inner diameter of the elongated tubular member is 700 microns or more.

3. The embolization microcatheter according to claim 1, wherein the thickness of the distal end of the wall is less than or equal to 120 microns.

4. The embolization microcatheter according to claim 1, further comprising a filter formed in the wall of the elongated tubular member, the filter comprising two or more filter segments, each filter segment comprising a plurality of side openings penetrating the wall, the plurality of side openings distributed circumferentially around the elongated tubular member.

5. The embolization microcatheter according to claim 4, wherein a proximal most of the filter segments comprises fewer side openings than rings distal thereto.

6. The embolization microcatheter according to claim 4, wherein a distal most of the filter segment is positioned 2-4 mm proximally to the distal end opening.

7. The embolization microcatheter according to claim 4, wherein side openings of the distal most filter segment are essentially square shaped.

8. The embolization microcatheter according to claim 7, wherein the essentially square shaped side openings have a dimension of 50×50 microns.

9. (canceled)

10. The embolization microcatheter according to claim 7, wherein the side openings of the remainder of the at least two filter segments are in a form of axial slits.

11. The embolization microcatheter according to claim 10, wherein the side openings of the remainder of the at least two filter segments have a width of 20 microns and wherein the length of the side openings of the remainder of the at least two filter segments varies between the side openings.

12. The embolization microcatheter according to claim 11, wherein the side openings of the remainder of the at least two filter segments have a length of at least 200 microns.

13. The embolization microcatheter according to claim 10, wherein at least some of the side openings of the remainder of the at least two filter segments have dimension of 2700×20 microns.

14. The embolization microcatheter according to claim 10, wherein at least some of the side openings of the remainder of the at least two filter segments have a dimension of 1350×20 microns.

15. The embolization microcatheter according to claim 10, wherein at least some of the side openings of the remainder of the at least two filter segments have a dimension of 300×20 microns.

16. The embolization microcatheter according to claim 1, wherein the braid is made of tungsten.

17. The embolization microcatheter according to claim 1, wherein the inner liner comprises Polytetrafluoroethylene (PTFE).

18. The embolization microcatheter according to claim 1, wherein the radiopaque marker comprises a metal marker band.

19. The embolization microcatheter according to claim 1, wherein the radiopaque marker is located 0.5-2 mm proximally to the distal end opening.

20. The embolization microcatheter according to claim 1, being suitable for delivery of embolization beads having a particle size of about 900 microns.

21. (canceled)

22. A method for delivering embolization beads, the method comprising:
delivering the embolization microcatheter of claim 1 to a target location via a delivery catheter; and
injecting beads through the microcatheter.

23.-26. (canceled)
